(19) United States
(12) Patent Application Publication
(10) Pub. No.: US 2006/0135946 A1
(43) Pub. Date: Jun. 22, 2006

(54) Hemostasis cuff for catheter securement

(75) Inventors: Ryan T. Moehle, Salt Lake City, UT (US); Ryan C. Patterson, Kaysville, UT (US); Kelly B. Powers, North Salt Lake City, UT (US); David M. Paolo, Cranston, RI (US); Alan L. Grumbling, Southampton, NJ (US)

Correspondence Address: MORRISON & FOERSTER, LLP, 555 WEST FIFTH STREET, SUITE 3500, LOS ANGELES, CA, 90013-1024, US

(73) Assignee: C. R. Bard, Inc., Murray Hill, NJ (US)

(21) Appl. No.: 11/297,703

(22) Filed: Dec. 8, 2005

Related U.S. Application Data
(60) Provisional application No. 60/639,051, filed on Dec. 21, 2004.

(51) Int. Cl. A61M 31/00

(52) U.S. Cl. 604/513; 604/175

(57) ABSTRACT

Implantable catheters including one or more cuffs are described herein. At least one of the cuffs may include a collagen material, such as Avitene® collagen. The catheter may include a combination of collagen cuffs and collagen-free polymeric cuffs. Various methods for the fabrication of collagen cuffs are also disclosed herein.

### CROSS-REFERENCE TO RELATED APPLICATIONS

This application claims the benefit under 35 U.S.C. § 119(e), to U.S. Provisional Application No. 60/639,051, filed Dec. 21, 2004, which is incorporated by reference into this application as if fully set forth herein.

### BACKGROUND OF THE INVENTION

Various approaches have been developed to facilitate hemostasis and/or to accelerate tissue ingrowth in order to improve the securement of an implanted catheter within the bodily tissue. In one common approach, a catheter cuff is provided along the length of the catheter to stabilize the position of the catheter within the implanted tissue. For example, the catheter cuff may serve as an interface for tissue ingrowth to stabilize the implanted catheter within a subcutaneous tunnel. The catheter cuff may also prevent infectious agents from migrating along the length of the catheter into a patient's body.

It is common to position a proximal portion of an implanted catheter in a subcutaneous tunnel to decrease the risk of infection that is associated with a catheter implant. Typically, a portion of the catheter is passed through a subcutaneous tunnel within a patient's body such that the catheter enters the body at a location that is displaced from the location where the catheter enters a major blood vessel within the patient's body. For example, a central line may be established by inserting a catheter into the subclavian vein that runs behind the clavicle, but the catheter entry point into the patient's body may be moved away from the area next to the clavicle to the area that is not immediately above the entry point into the subclavian vein. In this process, the actual access to the subclavian vein is still achieved by a puncture under the clavicle, but the proximal portion of the catheter is pulled under the skin through a subcutaneous tunnel to emerge from the body at a location, for example, close to the nipple. To secure the implanted catheter within the subcutaneous tunnel, a cuff is typically placed on the portion of the catheter that will be positioned within the subcutaneous tunnel. Post implantation, tissue surrounding the subcutaneous tunnel will grow into the cuff, thereby preventing the cuff from sliding within the subcutaneous tunnel. A cuff generally includes a plurality of large pores or indentations that may be utilized to allow ingrowth of tissue into the cuff structure to improve the securement of the catheter within the subcutaneous tunnel.

Although various catheter securement devices have been previously disclosed, it may be desirable to improve the current approaches by improving catheter and tissue interface. A cuff that provides hemostasis and/or accelerate tissue ingrowth may improve the clinical outcome of a catheter implant procedure and enhance the quality of patient care.

### BRIEF SUMMARY OF THE INVENTION

One aspect of the invention includes placing a collagen cuff along the portion of catheter to be positioned within the subcutaneous tunnel. The collagen cuff may be implemented in combination with a traditional polymeric cuff (e.g., polyester cuff, etc.). The collagen cuff (e.g., Avitene® collagen cuff, etc.) may facilitate hemostasis within the subcutaneous tunnel. In addition, the collagen cuff may also induce tissue ingrowth. When the collagen cuff is positioned next to a traditional polymeric cuff, the collagen may also induce tissue ingrowth around the traditional polymeric cuff. In another variation, the collagen cuff may be positioned close to the insertion site, where the implanted catheter enters the blood vessel. Various combinations of collagen and polymer cuff placement along the length of the portion of the catheter to be positioned within the subcutaneous tunnel are contemplated. In another variation, the collagen material is placed over a substantial portion of the catheter to be positioned within the subcutaneous tunnel.

In another aspect of the invention, a catheter cuff includes a combination of collagen and polymeric material is implemented to improve hemostasis and accelerate tissue ingrowth. For example, the cuff may include a polyester felt with embedded Avitene® collagen. In another variation, the cuff includes a polymeric base layer with interwoven Avitene® collagen filament. In yet another variation, the cuff includes two or more layers of materials. In one combination, an inner layer is free of collagen, while an outer layer includes a collagen material. A scrim or separation layer may be provided between the two layers to isolate the collagen material from the inner layer, or prevent the adhesive from the inner layer from encroaching the outer layer.

In one embodiment, a catheter securement device incorporates a plurality of cuffs positioned along a length of a catheter, including a first cuff configured for tissue ingrowth and a second cuff including a collagen material. In another embodiment, a catheter securement device incorporates a cuff having an inner layer and an outer layer positioned coaxially over the inner layer, the inner layer including a polymeric material and the outer layer including a polymeric material and a collagen material. In yet another embodiment, a method of creating a catheter securement device includes providing a polymeric member, incorporating a collagen material into the polymeric member; and forming the member into a cuff. In still another embodiment, a method of attaching a catheter securement device to a catheter includes disposing a hydrogel material over a desired region of a catheter, and positioning a polymeric cuff over the hydrogel material.

In one embodiment, a method for implanting a catheter includes placing a catheter including a cuff configured for tissue ingrowth in a subcutaneous tunnel, and releasing collagen into the subcutaneous tunnel from a location along the length of the catheter distal to the cuff. In another embodiment, a method of retarding the flow of blood from a wound includes providing a capsule containing a collagen material encapsulated within a dissolvable container and inserting the capsule into an open wound such that the collagen material is released from the container when the container dissolves.

These and other embodiments, features and advantages of the present invention will become more apparent to those skilled in the art when taken with reference to the following more detailed description of the invention in conjunction with the accompanying drawings that are first briefly described.

### BRIEF DESCRIPTION OF THE DRAWINGS

FIG. 1 illustrates an embodiment of a collagen sheet, which may be utilized for the fabrication of a collagen cuff. In this embodiment, a collagen foam is embedded in a top layer of a polymer sheet.

FIG. 2A illustrates an embodiment of a collagen sheet including a polymeric base layer with collagen filaments interwoven therein.

FIG. 2B illustrates another embodiment of a collagen sheet including a polymeric base layer with collagen filaments interwoven in a zigzag pattern.

FIG. 2C illustrates another embodiment of a collagen sheet including a polymeric base layer with collagen filaments interwoven in a crossover pattern.

FIG. 3 illustrates one variation of an implantable catheter with a dual cuff design, including a polymeric cuff and a collagen cuff.

FIG. 4 illustrates another variation of a dual cuff design, including a polymeric cuff and a collagen cuff.

FIG. 5 illustrates an implantable catheter with a dual cuff design implanted in a patient.

FIG. 6 illustrates an embodiment of an implantable catheter including fillet that forms a barrier to prevent the movement of a collagen cuff.

FIG. 7A illustrates one embodiment of a multi-cuff configuration, including a polymeric cuff and a collagen cuff.

FIG. 7B illustrates another embodiment of a multi-cuff, including a polymeric cuff and a collagen cuff.

FIG. 7C illustrates yet another embodiment of a multi-cuff configuration, including a polymeric cuff and two collagen cuffs.

FIG. 7D illustrates still another embodiment of a multi-cuff configuration, including two polymeric cuffs and a collagen cuff.

FIG. 8 illustrates one embodiment of a multi-cuff configuration, in which the cuffs are in contact with one another.

FIG. 9A illustrates anther embodiment of a single cuff design, including distinct sections of collagen.

FIG. 9B illustrates an embodiment of the single cuff design of FIG. 9A, in ections of collagen are supported by a base layer.

FIG. 10 illustrates an embodiment in which collagen material is placed along a of the catheter to be positioned within a subcutaneous tunnel.

FIG. 11A is a longitudinal cross-sectional view of an embodiment of a catheter g a dual layer cuff.

FIG. 11B is an axial cross-sectional view of the embodiment of FIG. 11A.

FIG. 12 is a longitudinal cross-sectional view of another embodiment of a including a multi-layer cuff, in which a separation layer is positioned between a layer and a polymeric base layer.

FIG. 13 illustrates an embodiment of a collagen sheet attached to a catheter.

### DETAILED DESCRIPTION OF THE INVENTION

The following detailed description should be read with reference to the drawings, in which identical reference numbers refer to like elements throughout the different figures. The drawings, which are not necessarily to scale, depict selected embodiments and are not intended to limit the scope of the invention. The detailed description illustrates by way of example, not by way of limitation, the principles of the invention. This description will clearly enable one skilled in the art to make and use the invention, and describes several embodiments, adaptations, variations, alternatives and uses of the invention, including what is presently believed to be the best mode of carrying out the invention.

Before describing the present invention, it is to be understood that unless otherwise indicated, this invention need not be limited to applications in humans. As one skilled in the art would appreciate, variations of the invention may be applied to various other animals as well. Moreover, it should be understood that embodiments of the present invention may be applied in combination with various fluid infusion and extraction devices for facilitating delivery and or extraction of fluids into a patient's circulatory system. For example, a drug pump may be utilized with the present invention to deliver medication into the patient's circulatory system. In another example, a dialysis machine may be utilized with the present invention to remove unwanted waste from the patient's circulatory system. In addition, one skilled in the art having the benefit of this disclosure would appreciate that the present invention may be implemented on a catheter having one or more lumens.

It must also be noted that, as used in this specification and the appended claims, the singular forms “a,” “an” and “the” include plural referents unless the context clearly dictates otherwise. Thus, for example, the term “a lumen” is intended to mean a single lumen or a combination of lumens, “a fluid” is intended to mean one or more fluids, or a mixture thereof. Furthermore, the words “proximal” and “distal” refer to directions closer to and away from, respectively, a physician operating the catheter, with the tip end (i.e., distal end) placed inside the patient's body. Thus, for example, the catheter end placed in the body of the patient would be the distal end of the catheter, while the catheter end outside the patient's body would be the proximal end of the catheter.

As used herein, the term “cuff” refers to a member configured to be positioned about an outer surface of a catheter or other medical device, as known to one skilled in the art. Various configurations are possible for a cuff, such as, for example, a cylindrical member, a cone-shaped member, a disc-shaped member, etc. The cuff embodiments described herein may include a collagen material, one of which is Avitene® collagen. Avitene® collagen is an active hemostat that physiologically interacts with the clotting mechanisms of a mammalian body to stop bleeding. The clotting mechanisms are enhanced by Avitene® collagen due to the micro-fibrillar structure thereof, which attracts and aggregates platelets. In one embodiment, Avitene® collagen can be described as a water insoluble partial hydrochloric acid salt of purified bovine corium collagen. Examples of various collagen-based materials that can be utilized to fabricate a collagen cuff are disclosed, for example, in U.S. Pat. No. 6,454,787 B1 to Maddalo et al., entitled “Collagen Hemostatic Foam” issued Sep. 24, 2002, and U.S. Pat. No. 6,361,551 B1 to Torgerson et al., entitled “Collagen Hemostatic Fibers” issued Mar. 26, 2002, each of which is incorporated herein by reference in its entirety as if fully set forth herein. Other biocompatible materials known to one skilled in the art that are suitable for fabrication of cuffs for disposition onto polymeric catheters are also contemplated. Also, a material such as a hydrogel (e.g., Tecophilic®, Tecogel®, etc.) may be used in conjunction with a polymeric cuff to provide, for example, hemostasis properties to the catheter. In a particular embodiment, the hydrogel swells upon exposure to fluids, such as mammalian body fluids, thereby preventing blood from exiting the tunnel. One or more cuffs may be disposed on a catheter to secure the catheter within a subcutaneous tunnel. The cuff or combination of cuffs may be referred to herein as a catheter securement device.

In one embodiment, a catheter securement device includes a cuff with collagen material, such as Avitene® collagen, which may accelerate tissue ingrowth into the cuff. The material properties of Avitene® collagen (e.g., sensitivity to heat) may prevent use of standard attachment methods of cuffs to catheters (e.g., utilizing heat and a glue bond), due to the possibility of such methods leading to denature of the collagen. Thus, inventive methods of attaching Avitene® collagen to a catheter are described herein. In certain embodiments, Avitene® collagen is attached directly to the catheter or is formed into a member that can be disposed over the catheter. In other embodiments, Avitene® collagen is incorporated into a polymeric cuff that is then attached to the catheter. Examples of these embodiments are provided below, although it should be appreciated that methods of implementing Avitene® collagen along a section of a catheter not specifically described herein are also within the scope of the invention.

First, with respect to attaching Avitene® collagen directly to a catheter, in one embodiment, an Avitene® collagen flour may be disposed onto a surface of the catheter. In one method, a biocompatible glue, which can also be biodegradable, such as cyanoacrylate, a UV-curable glue, epoxy, or other suitable adhesive, can be used to coat an outside surface of the catheter circumferentially and along a desired length, followed by rolling the region of the catheter having glue disposed thereon in the Avitene® collagen flour. In another method, the Avitene® collagen flour is dissolved in a solution, followed by dip coating a desired region of the catheter into the solution. When the solution precipitates, the Avitene® collagen will be on the desired region of the catheter. With respect to disposing an Avitene® collagen member onto a catheter, in one method, a process similar to powder metallurgy is utilized, in which Avitene® collagen flour is compacted into a mold to produce a cylindrical member that can be slid over a catheter shaft (e.g., the inside diameter of the member is about equal to the outside diameter of the catheter such that an interference fit is provided therebetween). In another method, the Avitene® collagen flour is molded onto a catheter using a two-piece mold, being compacted around an outer surface of the catheter along a desired region.

In another embodiment, an Avitene® collagen foam is used to create a cuff. In one method, the foam is attached to the catheter using an adhesive, such as cyanoacrylate. In another method, the foam is formed into either a cylindrical member or a cone-shaped member (either by cutting and shaping the foam or molding the foam) and slid over an outer surface of the catheter, as described above in connection with the compacted flour (e.g., the inside diameter of the member is sized to provide an interference fit over an outside diameter of the catheter). In another method, an Avitene® collagen foam with an adhesive backing is created such that after cutting to a proper size and shape, the formed member with adhesive backing can be positioned along a desired region of the catheter (e.g., the formed member is placed face down on a surface such that the adhesive backing is facing in an upward direction, followed by rolling the catheter over the backing to adhere the foam thereto).

In another embodiment, an Avitene® collagen mesh is used to create a cuff. In one method, the mesh is wrapped around the catheter shaft several times along a desired region. In another method, the mesh is formed into a member having either a cylindrical shape or a cone shape and is slid over an outer surface of the catheter. In a method combining the mesh with a polymeric cuff, the mesh is wrapped around a polymeric cuff, such as a PET cuff. In one variation, the mesh does not extend beyond the boundaries of the polymeric cuff. In another variation, a portion of the mesh extends beyond at least one edge of the polymeric cuff, the portion being tapered to the outer surface of the catheter shaft. Similarly, an Avitene® collagen foam could be combined with a polymeric cuff.

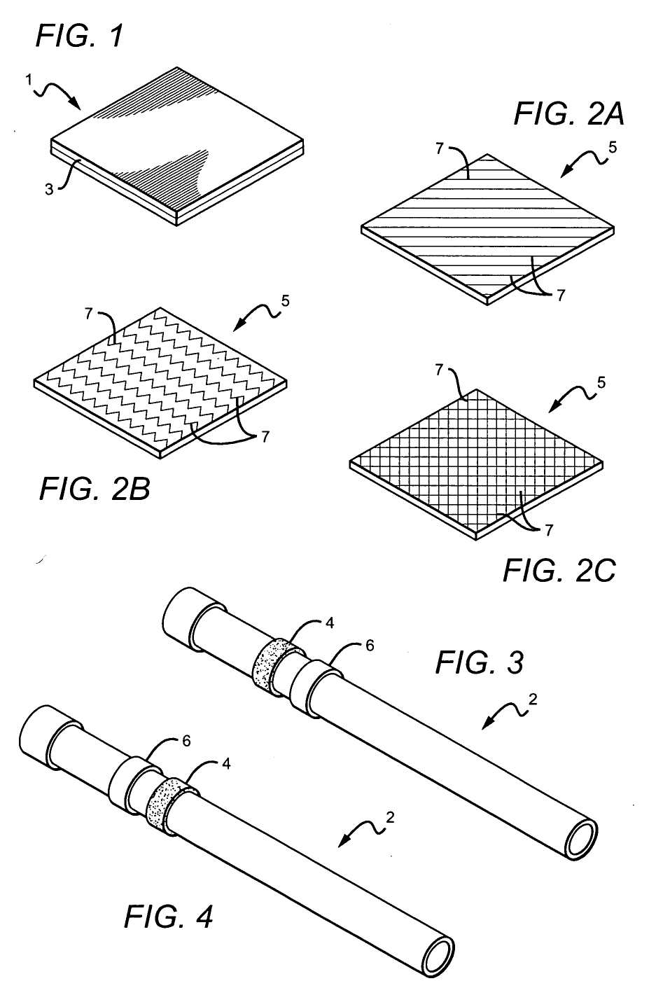

With respect to Avitene® collagen being incorporated into a polymeric material, in one embodiment, a cuff includes a polyester material embedded with Avitene® collagen. In one method, as shown in FIG. 1, a sheet of polyester felt 1 is enhanced by pouring an Avitene® collagen water slurry over it and then having the felt freeze-dried (lyophilized). The felt can be pre-wetted to induce the Avitene® collagen slurry to enter the polyester felt matrix. For example, the polyester felt may be pre-wetted with water prior to addition of the Avitene® collagen slurry. The polyester felt may also be agitated and mechanically stimulated to facilitate water incorporation. Following lyophilization, the polyester felt sheet 1, including Avitene® collagen incorporated into a top layer 3, may be cut to desired sizes. For example, the Avitene® collagen enhanced polyester felt sheet may be cut into a traditional polyester cuff size and then attached to the catheter using various methods well known to one skilled in the art (e.g., UV-curable adhesive, cyanoacrylate, etc.) In addition, a portion of the polymeric catheter tubing may be mechanically abraded prior to the placement of the collagen cuff to improve adhesion. In one example, a thin sheet (e.g., about 0.03 inches to about 0.05 inches) of polyester felt is utilized as a base layer and an Avitene® collagen water slurry is poured over the base layer and then freeze-dried. The Avitene® collagen water slurry may completely penetrate the polyester sheet, resulting in the Avitene® collagen being completely incorporated into the polyester sheet. In one variation, excess Avitene® collagen water slurry is provided such that an additional layer of Avitene® collagen matrix may form on top of the polyester base layer. In another variation, the appropriate amount of Avitene® collagen water slurry is provided such that all the Avitene® collagen is incorporated into the polyester layer, which results in no additional thickness.

In another method, which is similar to the freeze-dried method described above, the polyester felt is compressed during the freeze-dried process. The polyester felt is pre-wetted and then an Avitene® collagen slurry is poured over it as described above. The Avitene® collagen slurry is allowed to fully saturate the pre-wetted polyester felt. A compression mechanism is then implemented to apply pressure on the polyester felt during the freeze-dried process. For example, a sheet of plastic or metal may be placed over the saturated polyester felt to which pressure is applied (e.g., clamping, etc.) to provide even compression over the entire surface of the polyester felt. The polyester felt with saturated Avitene® collagen slurry is then freeze-dried. This approach may result in a thinner Avitene® collagen enhanced polyester felt material. The freeze-dried Avitene® collagen forms a matrix within the polyester felt which helps the polyester felt maintain a compressed state. After implantation and upon exposure to bodily fluids, the Avitene® collagen matrix within the polyester felt may lose a substantial portion of its material structural properties, such that the polyester felt may expand within the subcutaneous tunnel.

In another method, a plurality of openings or cavities is created in the polyester felt prior to the introduction of Avitene® collagen slurry. The openings in the polyester felt may improve adhesion of the Avitene® collagen to the polyester felt. In addition, the openings enable additional Avitene® collagen to be incorporated into the polyester felt. The incorporation of additional Avitene® collagen may improve hemostasis capabilities of the felt as well as the ability to accelerate tissue ingrowth. Furthermore, the openings filled with Avitene® collagen may be more prone to tissue ingrowth. One skilled in the art having the benefit of this disclosure would appreciate that various patterns or spatial configurations may be created in the polyester felt to improve the incorporation of Avitene® collagen and/or to provide enlarged orifices for tissue ingrowth. Such patterns or spatial configurations may completely penetrate through a thickness of polyester felt or may have dimensions that penetrate only partially through a thickness thereof.

In another embodiment, a sheet incorporating collagen is fabricated by interweaving polymer fibers with collagen fibers. For example, polyester fibers and Avitene® collagen fibers can be co-woven to form a structure that can be formed into a cuff. In one variation, the inner diameter of a formed cuff is configured such that there is a slight interference/friction with the catheter tubing. The outer circumference of the collagen cuff structure may be configured with an outer diameter that is similar to the outer diameter of a traditional polyester cuff. In one variation, the composition may be about 50% Avitene® collagen and about 50% polyester, although certainly any ratio of Avitene® collagen to polyester may be utilized. An increase in the ratio of Avitene® collagen composition may enhance tissue ingrowth and hemostasis. However, since Avitene® collagen tends to degrade substantially within a 4-6 week time period if not cross-linked, in some applications it may be advantageous to utilize more polyester than Avitene® collagen to maintain adequate bond strength to the catheter. In one variation, a combination of cross-linked and non-cross-linked collagen fibers are utilized for the fabrication of the cuff. Typically, cross-linking decreases hemostatic capabilities, but improves material properties and longer structural integrity.

In one embodiment, collagen filaments are interwoven into a polymeric felt or sheet to form a material layer that can be utilized to fabricate a collagen cuff. For example, a polyester felt is provided as a substrate layer and Avitene® collagen filaments, Avitene® collagen fibers, or Avitene® collagen yarn is then incorporated into the polyester felt (e.g., by using a sewing machine, etc.). Various sewing patterns may be utilized for the incorporation of the Avitene® collagen filaments. FIGS. 2A-2C illustrate examples of possible patterns. In FIG. 2A, collagen filaments 7 are interwoven into a polymeric base layer 5 as a plurality of parallel lines. In FIG. 2B, collagen filaments 7 are interwoven into the polymeric base layer 5 in a zigzag pattern. In FIG. 2C, collagen filaments 7 are interwoven into the polymeric base layer 5 in a crossover pattern. As the density of Avitene® collagen filaments within the polyester felt is increased, the likely result is an improvement in the hemostasis capabilities as well as the tissue ingrowth capabilities of the resulting collagen cuff. As discussed above, the Avitene® collagen filament enhanced polyester cuff may be attached to a polyurethane catheter shaft using an UV-curable adhesive, cyanoacrylate, epoxy, or other suitable adhesive, and other methods well known to one skilled in the art.

Referring now to FIG. 3, one embodiment of an implantable catheter 2 including a cuff 4 incorporating collagen, such as Avitene® collagen, which can be produced, for example, in any of the methods described above, is illustrated (hereinafter, “collagen cuff 4”). In this particular embodiment, collagen cuff 4 is positioned in close proximity to a polymeric cuff 6. “Close proximity” as used herein means the gap between adjacent cuffs or members is about 2 cm or less. The polymeric cuff 6 may include various biocompatible polymers that are well known to one skilled in the art. The polymeric cuff 6 may also include a material that facilitates the affixation of an implantable catheter 2 within the subcutaneous tunnel. In one variation, the polymeric cuff 6 includes a plug cuff made of polyester where the tissue does not grow into the cuff. The body tissue grows around the plug cuff and secures the catheter in place. In another variation, the polymeric cuff 6 is configured with pores and/or orifices for tissue ingrowth into the cuff structure, such that invasion of the living body tissue into the cuff may further secure the cuff within the subcutaneous tunnel. For example, the polymer cuff 6 may be polyester based, such as a Dupont Dacron® Cuff. In another variation, the polymer cuff may include a polyurethane sponge or polyethylene sponge.

Although in the example shown in FIG. 3, the collagen cuff 4 is positioned in close proximity to the polymeric cuff 6, one skilled in the art having the benefit of this disclosure would appreciate that the collagen cuff 4 may be positioned anywhere along the portion of the catheter to be placed within a formed subcutaneous tunnel. The collagen cuff 4 may be positioned on a distal side of the polymeric cuff 6, as shown in FIG. 4, rather than on the proximal side of the polymeric cuff 6, as shown in FIG. 3. It is also contemplated that two or more collagen and/or two or more polymeric cuffs may be positioned together on a catheter.

Placement of a collagen cuff 4 on the portion of the catheter positioned within a subcutaneous tunnel may facilitate hemostasis in the tissue surrounding the collagen cuff. In one variation, as a catheter is pulled through the subcutaneous tunnel, the collagen cuff may release a portion of the collagen material from the cuff and coat at least part of the tunnel surface with collagen materials. For example, the collagen cuff may include an Avitene® collagen foam. As the collagen cuff is passed through the subcutaneous tunnel, collagen material from the Avitene® collagen foam may sluff off or be drawn onto the tunnel surface due to friction between the outer surface of the cuff and the inner wall of the tunnel. Furthermore, the collagen cuff may accelerate tissue ingrowth onto the portion of the catheter surrounding the collagen cuff. For example, by placing the Avitene® collagen cuff in close proximity to the polymeric cuff, the collagen material from the collagen cuff may induce the ingrowth of the tissue onto the polymeric cuff.

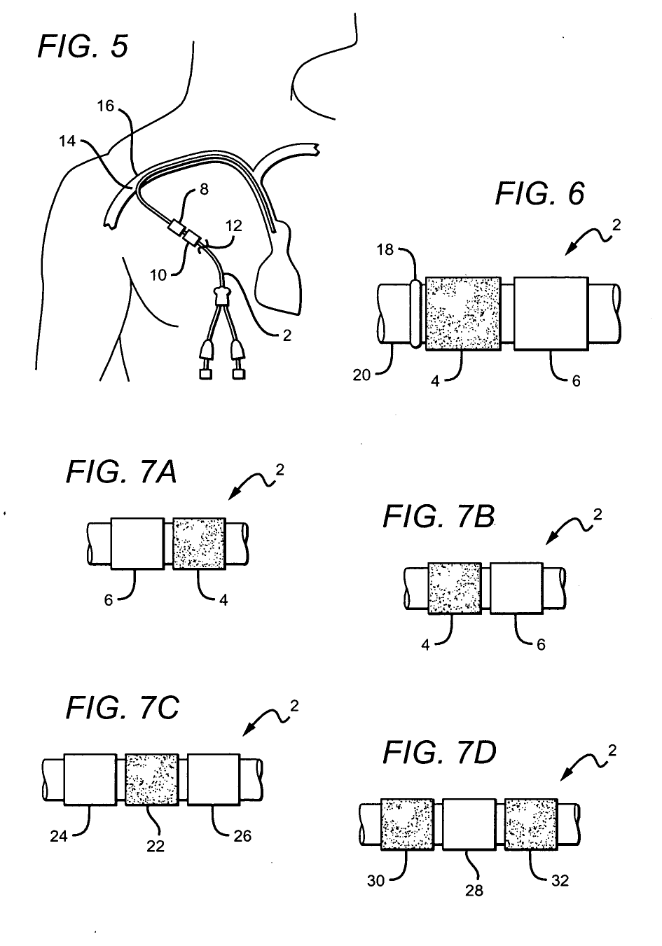

FIG. 5 illustrates one example of an implantable catheter 2. The collagen cuff can be placed in a variety of locations along the length of the subcutaneous tunnel. Typically, the length of the tunnel is between approximately six to ten centimeters. However, one skilled in the art would appreciate that subcutaneous tunnels of various lengths may be utilized depending on the particular medical procedure. In the example shown in FIG. 5, two cuffs 8, 10 are positioned adjacent a catheter exit site 12 (i.e., the catheter entry point into the patient's body). In one variation, the proximal cuff 10 is an Avitene® collagen cuff, while the distal cuff 8 is a polymeric cuff. In another variation, the proximal cuff 10 is a polymeric cuff, while the distal cuff is an Avitene® collagen cuff. In one variation, the two cuffs 8, 10 are positioned within a length of about 8 mm along the length of the implantable catheter 2.

The collagen cuff may also be positioned in close proximity to the blood vessel entry point 14 (i.e., where the catheter enters the circulatory system). Placement of a collagen cuff adjacent the blood vessel entry site 14 may induce wound healing in the punctured blood vessel 16 and the surrounding tissues, which may facilitate overall healing and decrease the incident of complications related to the catheter implant procedure. In another variation, two or more collagen cuffs are placed along the portion of the catheter positioned within the subcutaneous tunnel. For example, a collagen cuff may be placed close to the catheter exit site 12, while a second collagen cuff is placed close to the blood vessel entry site 14.

The collagen cuff may be attached to various intravenous catheters. For example, an Avitene® collagen cuff may be attached to a HemoSplit® dialysis catheter (C. R. Bard, Inc.) along with a typical polyester cuff, which can be inserted in the traditional fashion. The catheter is tunneled, and then the tips of the catheter are positioned in a blood vessel. In such a configuration, it may be beneficial to position the collagen cuff in close proximity to the polyester cuff to ensure compatibility independent of the tunnel length. In one embodiment, a collagen cuff (which may be similar in size to, or longer than, a polymeric cuff) is slidably positioned along a catheter shaft. The collagen cuff can be positioned in close proximity to the polyester cuff when packaged, but following tip placement, the doctor can slide the cuff to a position close to the entry site 14 on the blood vessel and then tunnel the proximal portion of the catheter. This would ensure that the collagen cuff is positioned close to catheter entry site 14 on the blood vessel. In one example in which a sliding cuff would be advantageous, a catheter implanted utilizing a reverse tunnel method may require a broad design tolerance in the placement of a collagen cuff. Because the blood vessel entry site 14 may be the greatest source of bleeding immediately post catheter implantation, it may be particularly beneficial to position the collagen cuff such that after implantation it is positioned close to the blood vessel entry site 14 (e.g., within about 3 cm from the blood vessel entry site).

The cuffs described herein may be attached directly to the catheter through various methods (e.g., ultraviolet (UV) cure, polyurethane glue, over-mold, interference fit, cyanoacrylate, etc.). In one example, an UV-curable adhesive, epoxy, and/or silicone adhesive, etc. fillet is utilized to trap the collagen cuff 14 between a fillet 18 and the polymeric cuff 6, as shown in FIG. 6. With this particular design, during tunneling any movement of the collagen cuff 4 would be impeded by either the polymeric cuff 6 or the fillet 18, depending on the direction of movement. UV-curable adhesive, cyanoacrylate, epoxy, or other suitable adhesive may be utilized for the creating the fillet 18 on the implantable catheter 2. UV cure polyurethane adhesive is inherently biocompatible and provides adequate bondability to polyurethane tubing. Utilizing a fillet 18 to prevent movement of the collagen cuff in a certain direction along the implantable catheter 2 avoids the potentially problematic step of heat-bonding the collagen cuff to the catheter, as discussed above.

For applications where reverse tunnel or retrograde tunneling is utilized, it may be desirable to position the adhesive fillet 18 at a distal point 20 along the implantable catheter 2 so that the collagen cuff 4 can be positioned deeper in the tunnel tract, effectively closer to the blood vessel entry site 14 (e.g., venous puncture site). In another variation, two fillets may be implemented, with one positioned proximal to a collagen cuff and the other positioned distal to the collagen cuff, in order to restrict movement of the collagen cuff along a predetermined length of the catheter. In one variation, a length is provided between the two fillets to allow the cuff to slide along a portion of the catheter. In another variation, the two fillets are positioned adjacent to and in contact with the cuff in order to fix the cuff at a designated position on the implantable catheter 2.

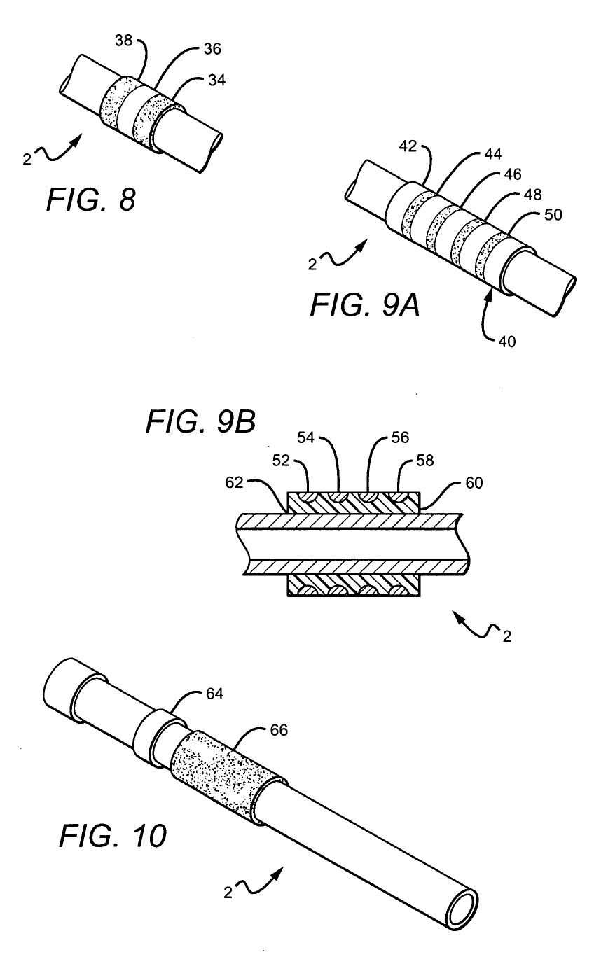

As discussed above, various combinations of collagen and polymeric cuffs may be implemented along the length of a catheter. For example, a collagen cuff 4 could be positioned distal to a polymeric cuff 6 along a length of a catheter (FIG. 7A), a collagen cuff 4 could be positioned proximal to a polymeric cuff 6 along a length of a catheter (FIG. 7B), a polymeric cuff 22 could be positioned between two collagen cuffs 24, 26 (FIG. 7C), a collagen cuff 28 could be positioned between two polymeric cuffs 30, 32, etc. Also, as one skilled in the art having the benefit of this disclosure would appreciate, four or more cuffs may be implemented with various combinations of patterns on an implantable catheter 2. It is also within the scope of the invention to position a plurality of collagen cuffs and polymeric cuffs next to one another such that the adjacent cuffs come into contact with one another. For example, FIG. 8 shows one example where three cuffs 34, 36, 38 are positioned along the length of the catheter, and each of them is connected to an adjacent cuff.

In one embodiment, a single cuff 40 may be configured with multiple regions or sections. Some regions can be embedded with collagen materials, while other regions are free of collagen materials. For example, in FIG. 9A, a cuff 40 including a polymeric base 42 is embedded with a plurality of bands 44, 46, 48, 50. Each of the bands 44, 46, 48, 50 can include a collagen material. The collagen bands 44, 46, 48, 50 may be configured such that they are as thick as the general thickness of the overall cuff 40. In another variation, the collagen bands 52, 54, 56, 58 may be incorporated into a polymeric base 60, such that they do not penetrate through base layer 62, and thus, do not come into direct contact with the catheter 2 surface, as illustrated in FIG. 9B.

In another embodiment, a collagen material is coated or layered along a portion of an implantable catheter such that when the catheter is implanted within a patient, a substantial portion (e.g., greater than 50%) of the catheter positioned within the subcutaneous tunnel is covered by the collagen material. An elongated collagen cuff or tubing may also be positioned along a mid-section of a catheter. FIG. 10 illustrates one example, in which an elongate portion of the catheter 2, distal to a polymer cuff 64, is coated with a collagen material 66 such that once the catheter 2 is implanted within a patient's body, the collagen material 66 is positioned within the subcutaneous tunnel. The length of implantable catheter 2 covered with the collagen material 66 in this example is approximately 4 cm, although one skilled in the art with the benefit of this disclosure would appreciate that various lengths of the catheter 2 could be covered.

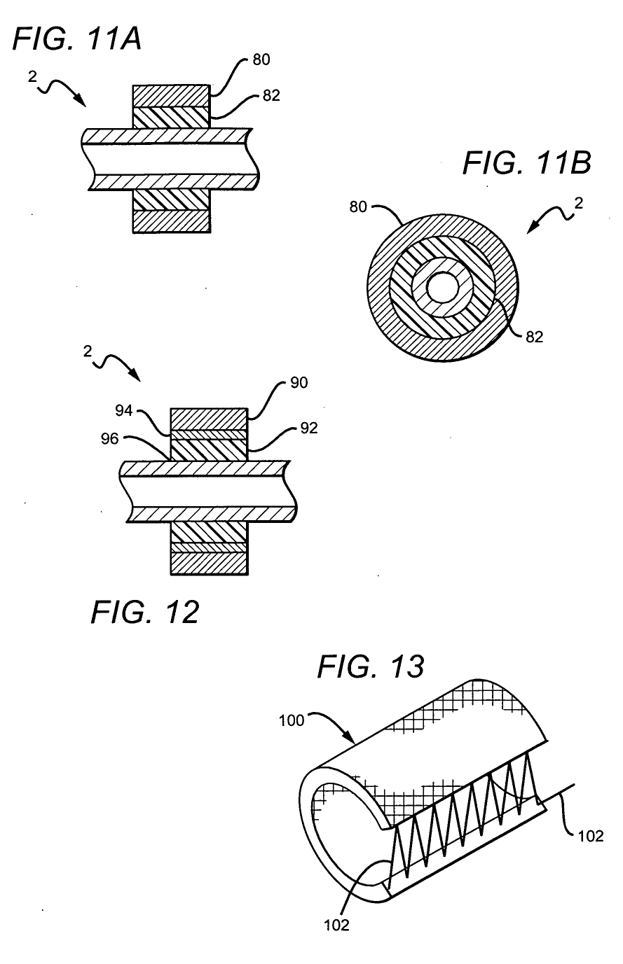

In another aspect of the invention, a collagen cuff is configured with a dual layer structure. In one embodiment, the cuff includes an outer layer including collagen and an inner layer that is substantially collagen-free (e.g., less than about 1% collagen by weight). In another embodiment, the inner layer does not contain any collagen. FIG. 11A illustrates one embodiment in which an outer layer 80 includes a collagen matrix and an inner layer 82 includes a polyester polymer. FIG. 11B is a cross-sectional view showing the collagen layer surrounding the polyester layer 82. After implantation, the collagen matrix begins to break down due to absorption of bodily fluid. As the tissue grows around the cuff, the collagen is absorbed by the body. Eventually a cuff with a smaller diameter than the originally implanted cuff remains. In another embodiment, the outer collagen layer 80 is embedded with a small amount of polymer to provide support to the collagen matrix (e.g., about 70% collagen and about 30% polymer). In another embodiment, both the outer 80 and the inner layer 82 include a polyester felt; however, only the outer layer is embedded with collagen.

In yet another embodiment, the inner layer 82 includes a non-resorbable polyester felt, while the outer layer 80 includes a resorbable polylactic/polyglycolic acid co-polymer felt, which may be incorporated with Avitene® collagen. After implantation, the collagen matrix within the outer layer starts to break down due to absorption of bodily fluid. The collagen material may improve hemostasis and enhance tissue ingrowth into the polymer cuff. The initial dual layers have a large diameter which may provide better security of the catheter within a subcutaneous tunnel. Once wound healing has taken place, the large diameter may no longer be necessary for securement. Over time, the polylactic/polyglycolic acid co-polymer layer is broken down and absorbed by the bodily tissue, leading to a cuff having a smaller diameter. The resulting cuff with a smaller diameter may allow easier extraction when, at a later time, the catheter is to be removed from the patient's body. Although in the above design only the outer layer 80 is incorporated with collagen, one skilled in the art would appreciate that both the outer and inner layer 82 maybe incorporated with collagen. For example, in another design, both the outer resorbable polymer layer and the inner non-resorbable polymer layer are embedded with collagen material.

In another embodiment, the cuff includes an outer layer 90, an inner layer 92, and a scrim 94 positioned between the outer and inner layer, as shown in FIG. 12. The scrim 94 is a layer of material that is configured to separate the outer layer 90 from the inner layer 92. The scrim 94 may include a thin film or a tightly woven polymeric sheath that serves as a barrier between the outer 90 and the inner 92 layers. For example, the scrim 94 may prevent adhesives that may be disposed on the inner base surface 96 of the inner layer 92 from contaminating the outer layer 90. The adhesive may permeate the inner layer, but once it reaches the scrim, it is blocked from further penetration. In this design, the integrity of a biological agent (e.g., collagen, growth factors, etc.) embedded within the outer layer 90 is protected from being contaminated by an adhesive.

In another embodiment, the outer layer 90 includes a non-woven polyester material that is configured to allow tissue ingrowth. The middle scrim layer 94 includes a woven polyester material. The inner layer 92 includes a non-woven polyester material. In this design, the scrim 94 may serve as a barrier to deep tissue ingrowth within the cuff. The scrim 94 may also block the adhesive from wicking into the outer layer of the cuff. In another design, a scrim layer is placed at the inner circumferential surface of the cuff serving as an interface to the catheter. For example, the cuff may include a first outer layer that includes a polyester material embedded with collagen and a second, inner layer, serving as a scrim. The inner layer is disposed on an inner circumferential surface of the outer layer and interfaces with the catheter body. An adhesive may be placed directly on the inner surface of the scrim to secure the cuff to the catheter. The scrim may be configured to prevent the adhesive from passing into the outer layer. In another variation, the polyester cuff with a scrim is configured with a thinner wall than the conventional polyester cuff commonly used in the industry, and thus may result in less trauma in the subcutaneous tunnel tract when it is removed from the body post implantation.

In another variation, the cuff includes multi-laminate layers of materials. In one example, the outer layer includes an absorbable non-woven fiber made of collagen (e.g., Avitene® collagen, etc.) or other bio-absorbable material (e.g., biocompatible absorbable polymer, etc.). The bio-absorbable material may be embedded with collagen. An optional middle layer comprising a carrier or scrim may be positioned therebetween. The scrim may serve as a support to attach the various layers and/or may serve as a barrier to the passage of adhesive from the bottom layer into the absorbable layer. An inner layer includes a non-absorbable material, such as a non-woven polyester fabric or other appropriate fibers/materials. The inner layer is left in the tissue after the outer layer is absorbed, serving to stabilize the catheter in the subcutaneous tunnel by acting as a plug. The scrim layer may also be configured to prevent ingrowth or to allow only a minimal amount of ingrowth, which permits the cuff to be removed with minimal effort on the part of the clinician.

The dual layer cuff (with or without the scrim) discussed above may be implemented by itself on the catheter or in combination with a traditional (i.e., collagen-free) polymeric cuff. One skilled in the art having the benefit of this disclosure would appreciate that the various collagen cuffs disclosed herein, whether single layered or multi-layered, may be implemented on a catheter by itself or in combination with other collagen and/or polymeric cuffs. In yet another variation, the cuff includes a single layer of absorbable material. In one design, the cuff includes an absorbable fiber made of collagen. In another design, the cuff includes a biocompatible absorbable polymer embedded with collagen. For example, biocompatible absorbable polymers that are well-known to one skilled in the art may be implemented. In applications utilizing only the absorbable cuff, once the cuff is absorbed by the surrounding tissue, the catheter can be easily removed from the patient's body.

As discussed above, various methods may be utilized to secure the collagen enhanced polymeric sheet onto a catheter. For example, adhesives may be used to attach a segment of the collagen embedded polymeric felt onto the catheter. In one approach, the cuff may be formed by gluing two ends of a polymeric strip to form a ring, and then inserting the ring over a catheter. The collagen enhanced polymeric ring may then be directly attached to the catheter body or be secured onto the catheter body with fillets. In another approach, a segment of a collagen enhanced polymeric sheet/felt may be wrapped around the catheter shaft and then secured onto the catheter by attaching the two free ends of the sheet/felt with rings or strings. For example, a pre-cut collagen enhanced sheet may be attached onto the shaft of a catheter with a corset-like approach. This approach may be particularly useful when the Avitene® collagen is in a mesh configuration. A sheet of collagen mesh 100 is pre-cut to the desired dimension. Tightening threads 102 are attached to the mesh sheet 100, such that the folded sheet is similar to a tube in construction, and forming a collagen cuff, as shown in FIG. 13. When the threads 102 are loose, the tube remains open and relatively compliant. When the tube is in the open/loss configuration, the surgeon can easily position the tube anywhere along the length of the catheter. To secure the tube on the catheter, the physician simply pulls on a single thread 102 that tightens the tube-like assembly and secures the collagen cuff to the catheter shaft.

In addition to a cuff configuration, Avitene® collagen can also be configured into a ring configuration to be positioned over a catheter to stop bleeding at the exit site 12 (FIG. 5). The ring may have a slit through one side to facilitate disposition onto the outer surface of the catheter. Similarly, Avitene® collagen rings may be configured for use with needles, micro-introducer devices, introducers, needle guides, or any other medical device as known to one skilled in the art. Moreover, an Avitene® collagen cuff could be positioned over a tunneler device and an Avitene® collagen pad could be incorporated into a surgical scalpel (e.g., the pad could have a slit through the middle for disposition over the blade of a scalpel). In one embodiment, the pad is rectangular shaped and is disposed perpendicular to the blade of the scalpel. Moreover, Avitene® collagen material (e.g., powder, fabric, etc.) can be compressed and packaged in a gel or other fast-dissolving capsule container. The Avitene® collagen capsule can then be inserted into a wound, where the capsule container would quickly dissolve to release the Avitene® collagen material and thereby rapidly close the wound. For example, Avitene® collagen mesh encapsulated within the container would provide a physical barrier upon release into the wound.

As mentioned above, in another embodiment, a hydrogel material, such as Tecophilic® or Tecogel®, may be used in conjunction with a polymeric cuff. In one embodiment, a polymeric cuff, such as a polyester cuff, is attached to a desired region of a catheter using a glue band of hydrogel material. The cuff may be slit in select locations to accommodate swelling of the hydrogel upon introduction into a patient's body. The hydrogel in this embodiment provides, for example, instant cuff fixation to prevent movement of the catheter within a subcutaneous tunnel, hemostasis as the hydrogel absorbs bodily fluids, and improved tissue ingrowth as the swelling presses the cuff into the bodily tissue. In another embodiment, a cuff formed of a hydrogel material is disposed along a region of a catheter adjacent a polymeric cuff. The hydrogel cuff could be configured similarly to the polymeric cuff (e.g., cylindrically shaped) or could be cone-shaped or tapered to facilitate catheter insertion when the hydrogel cuff is positioned distally of the polymeric cuff (i.e., the hydrogel comes in contact first with the subcutaneous tunnel upon insertion). The hydrogel cuff swells upon contact with bodily fluids to fix the catheter in position. Of course, the positioning and configuration of the hydrogel cuff could be the same as or similar to any described herein with respect to the collagen cuff.

This invention has been described and specific examples of the invention have been portrayed. While the invention has been described in terms of particular variations and illustrative figures, those of ordinary skill in the art will recognize that the invention is not limited to the variations or figures described. In addition, where methods and steps described above indicate certain events occurring in certain order, those of ordinary skill in the art will recognize that the ordering of certain steps may be modified and that such modifications are in accordance with the variations of the invention. Additionally, certain of the steps may be performed concurrently in a parallel process when possible, as well as performed sequentially as described above. Therefore, to the extent there are variations of the invention, which are within the spirit of the disclosure or equivalent to the inventions found in the claims, it is the intent that this patent will cover those variations as well. Finally, all publications and patent applications cited in this specification are herein incorporated by reference in their entirety as if each individual publication or patent application were specifically and individually put forth herein.

What is claimed as new and desired to be protected by Letters Patent of the United States is:

1. A catheter securement device, comprising a plurality of cuffs positioned along a length of a catheter, including a first cuff configured for tissue ingrowth and a second cuff including a collagen material.

2. The catheter securement device according to claim 1, wherein the collagen material comprises water soluble partial hydrochloric acid salt of purified bovine corium collagen.

3. The catheter securement device according to claim 2, wherein the form of the collagen material includes flour, foam, mesh, slurry, fibers, filaments, or yarn and combinations thereof.

4. The catheter securement device according to claim 1, wherein the plurality of cuffs comprises a first polymeric cuff and a second collagen cuff.

5. The catheter securement device according to claim 4, wherein the first polymeric cuff is positioned in close proximity to the second collagen cuff.

6. The catheter securement device according to claim 4, wherein the first polymeric cuff is in contact with the second collagen cuff.

7. The catheter securement device according to claim 4, wherein the second collagen cuff is positioned distally of the first polymeric cuff.

8. The catheter securement device according to claim 4, wherein the first polymeric cuff comprises a polyester material.

9. The catheter securement device according to claim 4, further comprising a third polymeric cuff, wherein the second collagen cuff is positioned between the first and third polymeric cuffs.

10. The catheter securement device according to claim 4, wherein the second collagen cuff is slidably positioned on the catheter.

11. The catheter securement device according to claim 10, further comprising a fillet, wherein the second collagen cuff is positioned between the fillet and the first polymeric cuff.

12. A catheter securement device, comprising a cuff including an inner layer and an outer layer positioned coaxially over the inner layer, the inner layer including a polymeric material and the outer layer including a polymeric material and a collagen material.

13. The catheter securement device according to claim 12, wherein the inner layer comprises a polyester felt and the outer layer comprises a collagen foam.

14. The catheter securement device according to claim 12, wherein the outer layer polymeric material is absorbable.

15. A catheter securement device, comprising a polymeric cuff including a plurality of bands, wherein each of the bands includes a collagen material.

16. A method for implanting a catheter, comprising:
placing a catheter including a cuff configured for tissue ingrowth in a subcutaneous tunnel; and
releasing collagen into the subcutaneous tunnel from a location along the length of the catheter distal to the cuff.

17. A method of creating a catheter securement device, comprising:
providing a polymeric member;
incorporating a collagen material into the polymeric member; and
forming the member into a cuff.

18. The method according to claim 17, wherein the collagen material is a collagen mesh, and wherein the incorporating includes wrapping the mesh about the cuff.

19. The method according to claim 17, wherein the polymeric member is a sheet of polyester felt and the collagen material is a water soluble partial hydrochloric acid salt of purified bovine corium collagen slurry, the incorporating comprising pre-wetting the felt and pouring the slurry onto the pre-wetted felt.

20. The method according to claim 19, wherein the incorporating further comprises freeze-drying the felt following the pouring.

21. The method according to claim 20, wherein the freeze-drying comprises compressing the felt.

22. The method according to claim 19, wherein the incorporating further comprises creating a plurality of cavities in the felt prior to pouring.

23. The method according to claim 17, wherein the polymeric member is a sheet of polyester felt and the collagen material includes a plurality of filaments, fibers or yarn, and wherein the incorporating comprises sewing the filaments, fibers or yarn into the felt.

24. A method of retarding the flow of blood from a wound, comprising providing a capsule containing a collagen material encapsulated within a dissolvable container and inserting the capsule into an open wound such that the collagen material is released from the container when the container dissolves.

25. A method of attaching a catheter securement device to a catheter, comprising:
disposing a hydrogel material over a desired region of a catheter; and
positioning a polymeric cuff over the hydrogel material.

26. The method according to claim 25, further comprising creating slits in the polymeric cuff.
